(19) United States
(12) Patent Application Publication
(10) Pub. No.: US 2024/0075277 A1
(43) Pub. Date: Mar. 7, 2024

(54) CARDIAC SUPPORT SYSTEM INLETS AND CONNECTING DEVICES

(71) Applicant: KARDION GMBH, Stuttgart (DE)

(72) Inventors: Inga Schellenberg, Stuttgart (DE); Mario Heintze, Stuttgart (DE); Hardy Baumbach, Stuttgart (DE); Johannes Bette, Balingen (DE); Marvin Mitze, Stuttgart (DE)

(21) Appl. No.: 18/461,385

(22) Filed: Sep. 5, 2023

Related U.S. Application Data
(60) Provisional application No. 63/374,684, filed on Sep. 6, 2022.

(51) Int. Cl. A61M 60/148; A61M 60/268; A61M 60/837

(52) U.S. Cl. CPC A61M 60/148; A61M 60/268; A61M 60/837

(57) ABSTRACT

Inlet device and connecting devices for a minimally invasive miniaturized percutaneous mechanical circulatory support system. The inlet device includes an inlet portion and a transfer portion with a support structure. The inlet device can be used for transmitting a body fluid of a patient, for example blood, to an impeller of a pump of the circulatory support system. The connecting device can include a receiving element and an insertion element. The receiving element of the connecting device can include a receiving structure that the insertion element of the connecting device can be pushed into. The insertion element can include at least one slide-on ramp, the slide-on ramp being connectable to the receiving structure in a form-fitting, non-positive, force-locking, and/or self-locking manner. The inlet device can include a receiving element or an insertion element of the connecting device.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims the priority benefit of U.S. Provisional Patent Application No. 63/374,684, filed Sep. 6, 2022, the entire disclosure of which is hereby incorporated by reference herein in its entirety.

### BACKGROUND

### Field

The technology relates generally to mechanical circulatory support systems, in particular to inlets and connecting devices for such systems.

### Description of the Related Art

Mechanical circulatory support systems are used to assist with pumping blood. Such pumping may be useful in various contexts. For example, percutaneous coronary intervention (PCI) is a non-surgical procedure to revascularize stenotic coronary arteries. PCI includes a variety of techniques, e.g. balloon angioplasty, stent implantation, rotablation and lithotripsy. A PCI is considered high risk if either the patient has relevant comorbidities (e.g. frailty or advanced age), the PCI per se is very complex (e.g. bifurcation or total occlusions) or hemodynamic status is challenging (e.g. impaired ventricular function). Mechanical circulatory support systems may be used to assist with pumping blood during these and other procedures. Conventional systems are complex and difficult to use, requiring complex components and processes.

There remains a need for a temporary and/or long term mechanical circulatory support system with simpler features and components and that overcomes the aforementioned and other drawbacks.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for circulatory support systems.

In one aspect, a minimally invasive miniaturized percutaneous mechanical circulatory support system is provided. The system may be placed across the aortic valve via a single femoral arterial access point. The system includes a low profile axial rotary blood pump carried by the distal end of an eight French catheter. The system can be percutaneously inserted through the femoral artery and positioned across the aortic valve into the left ventricle. The device actively unloads the left ventricle by pumping blood from the left ventricle into the ascending aorta and systemic circulation. The system may include an inlet device and/or a connecting device as described herein.

In another aspect, a mechanical circulatory support system for high risk coronary interventions and use thereafter may be provided. The system may include an elongate flexible catheter shaft, having a proximal end and a distal end, a circulatory support device carried by the distal end of the shaft, the circulatory support device including a tubular housing, having a proximal end and a distal end, an impeller within the housing, a removable guidewire guide tube entering a first guidewire port on a distal end of the housing, exiting the housing via a second guidewire port on a side wall of the housing distal to the impeller, reentering the housing via a third guidewire port on a proximal side of the impeller, and extending proximally into the catheter shaft. The system may include a motor within the housing and configured to rotate the impeller. The motor may be positioned distal to the third guidewire port. The tubular housing may an axial length in a range of 60 mm to 100 mm. The system may include a blood exit port on the tubular housing in communication with the impeller, and a blood inlet port spaced distally apart from the blood exit port. The housing may include a flexible slotted tube covered by an outer polymeric sleeve, an inner polymeric sleeve, or both and inner and an outer polymeric sleeve. The housing with the blood inlet port may comprise an inlet device. The system may include a sealed motor housing inside of the tubular housing. The system may include a connecting device as described herein.

In another aspect, a mechanical circulatory support system for high risk coronary interventions and support use thereafter may be provided. The system may include a circulatory support catheter, including a circulatory support device carried by an elongate flexible catheter shaft, an insertion tool having a tubular body and configured to axially movably receive the circulatory support device, and an access sheath, having a tubular body and configured to axially movably receive the insertion tool. The access sheath may include an access sheath hub having an insertion tool lock for engaging the insertion tool. The access sheath hub may include a catheter shaft lock for locking the access sheath hub to the catheter shaft. The circulatory support device may include an inlet device and/or connecting device as described herein.

In another aspect, an inlet device for guiding a mechanical circulatory support system and/or for transferring a body fluid of a patient, for example blood of a patient, may be provided. The inlet device may be used for transmitting blood of a patient to an impeller of a pump of a circulatory support system. The inlet device may span an aortic valve of a patient and be used to transmit blood from a left ventricle of a heart of a patient to an impeller of a blood pump of a circulatory support system that resides in an ascending aorta, an aortic arch, or an aorta of the patient. The inlet device may be deformably formed and comprised of a super-elastic material (for example, Nitinol). The inlet device may comprise an inlet portion and a transfer portion. The inlet device may comprise an inlet portion and a transfer portion with one or more transition portions, with a cross-sectional area and/or diameter of the inlet portion less than that of the transfer portion. The inlet device may include a connection device or an element thereof as described herein.

In another aspect, a connecting device and method for connecting components of a mechanical circulatory support system may be provided. In some embodiments, the connecting device may be used for connecting an inlet device to an impeller housing of a mechanical circulatory support system. In some embodiments, the connecting device may be used for connecting an inlet device, which may include an impeller housing, to a motor housing of a mechanical circulatory support system. In some embodiments, the connecting device may be used for connecting a component comprising a Nitinol tubular structure, for example, an inlet device, to a component comprising titanium, for example, an impeller housing or a motor housing. The connecting device may include a receiving element and an insertion element. The receiving element of the connecting device may include a receiving structure that the insertion element of the connecting device can be pushed into. The insertion element may include at least one slide-on ramp, the slide-on ramp being connectable to the receiving structure in a form-fitting, non-positive, force-locking, and/or self-locking manner. The connecting device may be used to transmit forces, for example, tensile, compressive, and torsional forces. In some embodiments, an inlet device of a mechanical circulatory support system may comprise a receiving element of a connecting device on its proximal end, and an impeller housing may comprise an insertion element on its distal end for connecting to the receiving element. In some embodiments, an inlet device of a mechanical circulatory support system may comprise an impeller housing and a receiving element of a connecting device on its proximal end, and a motor housing may comprise an insertion element on its distal end for connecting to the receiving element. In some embodiments, an inlet device of a mechanical circulatory support system may comprise an insertion element of a connecting device on its proximal end, and an impeller housing may comprise a receiving element on its distal end for connecting to the insertion element. In some embodiments, an inlet device of a mechanical circulatory support system may comprise an impeller housing and an insertion element of a connecting device on its proximal end, and a motor housing may comprise a receiving element on its distal end for connecting to the insertion element.

In another aspect, a connecting device and method for attaching and/or detaching a component of a mechanical circulatory support system may be provided. The connecting device may include a receiving element and an insertion element. The receiving element of the connecting device may include a receiving structure that the insertion element of the connecting device can be pushed into. The insertion element may include at least one slide-on ramp, the slide-on ramp being connectable to the receiving structure in a form-fitting, non-positive, force-locking, and/or self-locking manner. The connecting device may be used to transmit forces, for example, tensile, compressive, and torsional forces.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawing, can be arranged, substituted, combined, and designed in a wide variety of different configurations, all of which are explicitly contemplated and make part of this disclosure.

FIG. 1 is a cross-sectional view of a distal end of a mechanical circulatory support (MCS) system supported by a catheter and positioned across an aortic valve according to some embodiments.

FIG. 2 schematically illustrates an MCS system inserted into the body via the access pathway from the femoral artery to the left ventricle according to some embodiments.

FIG. 3 is a side elevational view of an embodiment of an MCS system that may incorporate the various features described herein.

FIG. 4 shows the system of FIG. 3, with the introducer sheath removed and including an insertion tool and a guidewire loading aid according to some embodiments.

FIG. 5 is a side view of an embodiment of an introducer kit, having a sheath and dilator, and that may be used with the various MCS systems and methods described herein.

FIG. 6 shows an embodiment of a placement guidewire that may be used with the various MCS systems and methods described herein.

FIG. 7 is a perspective view of a distal pump region of the MCS system shown in FIG. 1 according to some embodiments.

FIG. 8 is a side cross-section view of a distal region of the MCS system shown in FIG. 1, showing the guidewire path and the guidewire loading aid in place according to some embodiments.

FIG. 9A is a side view of an embodiment of an MCS device that may be used with the MCS system shown in FIG. 1 and according to some embodiments.

FIG. 9B is a partial cross-sectional view of the MCS device of FIG. 9A showing an embodiment of a seal according to some embodiments.

FIG. 10 is a side view of an inlet device according to some embodiments.

FIG. 11 is a side view of a support structure of an inlet device according to FIG. 10 and to some embodiments.

FIG. 12 is a detailed view of a winding region of an inlet device according to some embodiments.

FIG. 13 is a detailed view of a first engagement region of an inlet device according to some embodiments.

FIG. 14 is a detailed view of a second engagement region of an inlet device according to some embodiments.

FIG. 15 is a detailed view of a connection region of an inlet device according to some embodiments.

FIG. 16 is a side view of an inlet device according to some embodiments.

FIG. 17 shows a support structure of an inlet device according to FIG. 16 and to some embodiments.

FIG. 18 is a detailed view of a finger engagement region of an inlet device according to some embodiments.

FIG. 19 is a side view of an inlet device according to some embodiments.

FIG. 20 is a perspective view of a connecting device according to some embodiments.

FIG. 21 is a front end view of a connecting device according to some embodiments.

FIG. 22 is a side view of a connecting device according to some embodiments.

FIG. 23 is a cross-sectional view of a connecting device according to some embodiments.

FIG. 24 is a detailed, partial cross-sectional view of the connecting device shown in FIG. 23 according to some embodiments.

FIG. 25 is a side view of an inlet device with inlet and outlet openings according to some embodiments.

FIG. 26 is a side view of the inlet device of FIG. 25 including a coating according to some embodiments.

FIG. 27 shows a support of the inlet device of FIG. 25 according to some embodiments.

FIG. 28 shows a side view of a wound inlet device of FIG. 27 according to some embodiments.

While the above-identified drawings set forth presently disclosed embodiments, other embodiments are also contemplated, as noted in the detailed description. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

A mechanical circulatory support (MCS) system as described herein may include a temporary (e.g., generally no more than about 6 hours, or no more than about 3 hours, 4 hours, 5 hours, 7 hours, 8 hours, or 9 hours) left ventricular support device for use during various procedures, such as high-risk percutaneous coronary intervention (PCI) performed in elective or urgent, hemodynamically stable patients with severe coronary artery disease and/or depressed left ventricular ejection fraction. The system may be used if a heart team, including a cardiac surgeon, has determined high risk PCI is the appropriate therapeutic option. Alternatively, the MCS system as described herein may include a long-term left ventricular support device for use during and/or after high-risk PCI performed in elective or urgent, hemodynamically stable patients with severe coronary artery disease and/or depressed left ventricular ejection fraction, when a heart team, including a cardiac surgeon, has determined high risk PCI is the appropriate therapeutic option. Alternatively, the MCS system as described herein may include a long-term left ventricular support device for use in patients when a health care provider has determined it is an appropriate therapeutic option. The embodiments of MCS systems and devices as described herein may be placed across the aortic valve, for example via a single femoral arterial access.

The system 10 may include a low-profile axial rotary blood pump mounted on a catheter such as an 8 French (Fr) catheter, referred to as an MCS pump or MCS device. When in place, the MCS pump can be driven by an MCS controller to provide up to about 4.0 liters/minute of partial left ventricular support, at about 60 mm Hg. No system purging is needed due to improved bearing design and sealed motor, and the system is visualized fluoroscopically eliminating the need for placement using sensors.

The system may further include an expandable sheath, which allows 8-10 Fr initial access size for easy insertion and closing, expandable to allow introduction of 14 Fr and 18 Fr pump devices and return to a narrower diameter around the 8 Fr catheter once the pump has passed. This feature may allow passage of the heart pump through vasculature while minimizing shear force within the blood vessel, advantageously reducing risk of bleeding and healing complications. Distention or stretching of an arteriotomy may be done with radial stretching with minimal shear, which is less harmful to the vessel. Access may be accomplished via transfemoral, transaxillary, transaortal, transapical approach, or others.

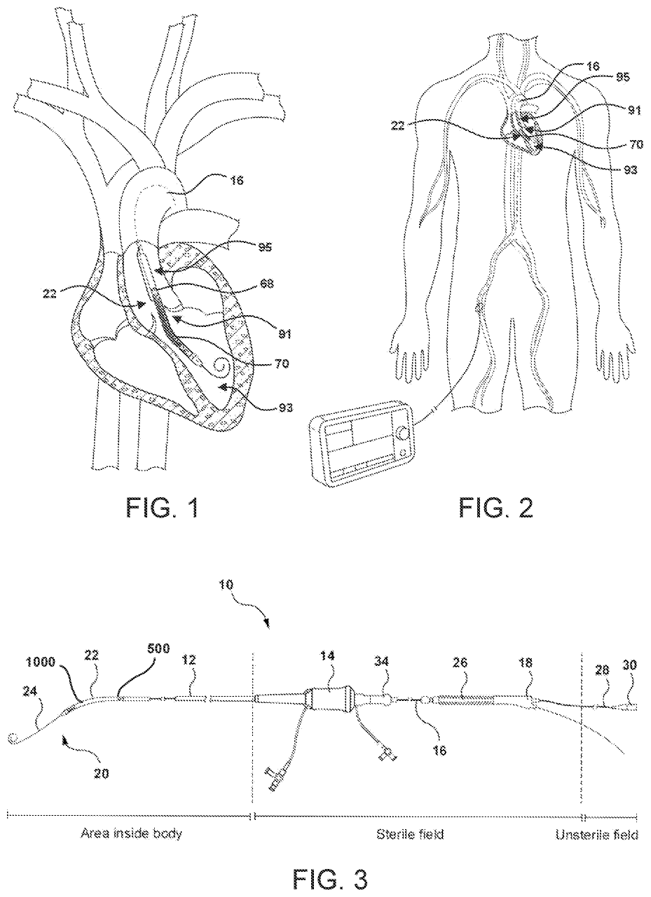

FIG. 1 shows a distal end of an embodiment of an MCS system 10 having a pump 22 mounted on the tip of an 8 Fr catheter 16. An inlet tube portion 70 of the device extends across the aortic valve 91. In some embodiments, the inlet tube portion comprises an inlet device as described herein. An impeller is located at the outflow section of the inlet tube drawing blood from the left ventricle 93 through the inlet tube portion 70 and ejecting it out the outflow section 68 into the ascending aorta 95. The motor is mounted directly proximal to the impeller in a sealed housing eliminating the need to flush the motor prior to or during use. This configuration provides hemodynamic support during high-risk PCI, time and safety for a complete revascularization via a minimally invasive approach (rather than an open surgical procedure).

The system has been designed to eliminate the need for motor flushing or purging, and to provide increased flow performance up to 4.0 l/min at 60 mmHg with acceptably safe hemolysis due to a computational fluid dynamics (CFD) optimized impeller that minimizes shear stress.

The MCS Device actively unloads the left ventricle by pumping blood from the ventricle into the ascending aorta and systemic circulation (shown in FIGS. 1 and 2). When in place, the MCS device can be driven by a complementary MCS Controller to provide between 0.4 l/min up to 4.0 l/min of partial left ventricular support.

In general, the overall MCS system 10 may include a series of related subsystems and accessories, including one or more of the following:
The MCS Device may include a pump, shaft, proximal hub, insertion tool, proximal cable, infection shield, guidewire aid, inlet device, and connecting device. The MCS Device may be provided sterile;
The MCS shaft may contain the electrical cables and a guidewire lumen for over-the-wire insertion. The MCS shaft may also include a connecting device;
The proximal hub may contain a guidewire outlet with a valve to maintain hemostasis and connects the MCS shaft to the proximal cable, that connects the MCS Device to the MCS Controller;
The proximal cable may be 3.5 m (approximately 177 inch) in length and extend from the sterile field to the non-sterile field where the MCS Controller is located;
An MCS insertion tool may be part of the MCS Device to facilitate the insertion of the pump into an Introducer Sheath and to protect the inlet tube and the valves from potential damage or interference when passing through the Introducer Sheath;
A peel-away guidewire aid may be pre-mounted on the MCS Device to facilitate the insertion of the 0.018″ placement guidewire into the pump and into the MCS shaft;
A 3 m long, 0.018″ diameter placement guidewire may be used, having a soft coiled pre-shaped tip for atraumatic wire placement into the left ventricle. The guidewire may be provided sterile;
A 14 Fr Introducer Sheath with a usable length of 275 mm may be used to maintain access into the femoral artery and provide hemostasis for the 0.035″ guidewire, the diagnostic catheters, the 0.018″ placement guidewire, and the insertion tool. The housing of the Introducer Sheath may be designed to accommodate the MCS Insertion Tool. The Introducer Sheath may be provided sterile;
An introducer dilator may be compatible with the Introducer Sheath to facilitate atraumatic insertion of the Introducer Sheath into the femoral artery. The introducer dilator may be provided sterile; and/or
An MCS Controller may drive and/or operate the MCS Device, observes its performance and condition as well as provide error and status information. The powered controller may be designed to support at least about 12 hours of continuous operation or may be configured for long-term use and may contain a basic interface to indicate and adjust the level of support provided to the patient. Moreover, the controller may provide an optical and audible alarm notification in case the system detects an error during operation. The MCS Controller may be provided non-sterile and can be contained in an enclosure designed for cleaning and re-use outside of the sterile field. The controller enclosure may contain a socket into which the extension cable is plugged.

Referring to FIG. 3, there is illustrated an overall MCS system 10 in accordance with some embodiments, subcomponents of which will be described in greater detail below. The system 10 may include an introducer sheath 12 having a proximal introducer hub 14 with a central lumen for axially movably receiving an MCS shaft 16. The MCS shaft 16 may extend between a proximal hub 18 and a distal end 20. The hub 18 may be provided with an integrated microcontroller or memory storage device for device identification and tracking of the running time, which could be used to prevent overuse to avoid excessive wear or other technical malfunction. The microcontroller or memory device could disable the device, for example to prevent using a used device. They could communicate with the controller, which could display information about the device or messages about its usage. An atraumatic cannula tip with radiopaque material allows the implantation/explantation to be visible under fluoroscopy.

A pump 22 may be carried by a distal region of the MCS shaft 16. Pump 22 may comprise an inlet device 1000 at its distal end. The system 10 may be provided with at least one central lumen for axially movably receiving a guide wire 24. The proximal hub 18 may additionally be provided with an infection shield 26. A proximal cable 28 may extend between the proximal hub 18 and a connector 30 for releasable connection to a control system typically outside of the sterile field, to drive the pump 22.

A connecting device 500 may be located near a proximal end and/or a distal end of the pump 22. The connecting device may be used for connecting components of the pump 22, for example, an inlet device 1000 to an impeller housing and/or to a motor housing of pump 22.

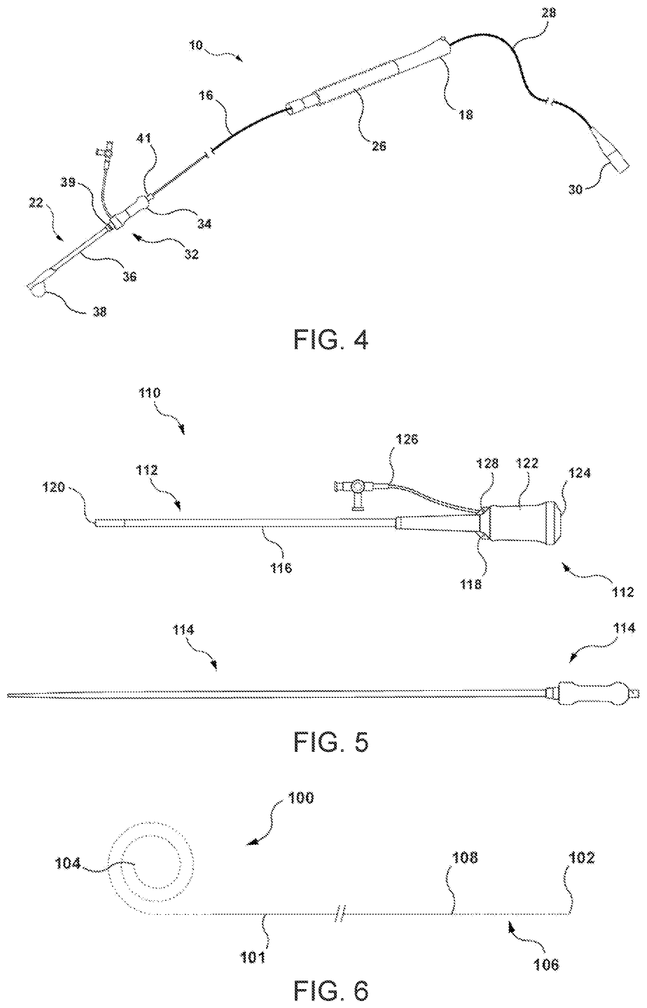

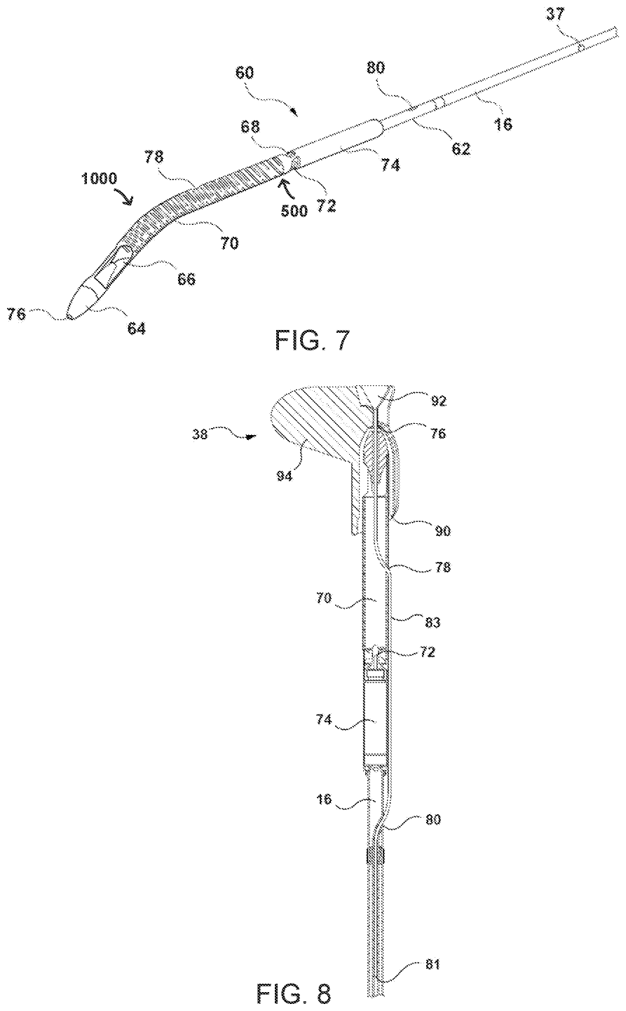

Referring to FIG. 4, the system 10 may additionally include an insertion tool 32, having an elongate tubular body 36 having a length within the range of from about 85 mm to about 160 mm (e.g., about 114 mm) and an inside diameter within the range of from about 4.5 mm to about 6.5 mm (e.g., about 5.55 mm), extending distally from a proximal hub 34. The tubular body 36 includes a central lumen adapted to axially movably receive the MCS shaft 16 and pump 22 there through, and sufficient collapse resistance to maintain patency when passed through the hemostatic valves of the introducer sheath. As illustrated in FIG. 4, the pump 22 can be positioned within the tubular body 36, such as to facilitate passage of the pump 22 through the hemostatic valve(s) on the proximal end of an introducer hub 14. A marker 37 (FIG. 7) is provided on the shaft 16 spaced proximally from the distal tip 64 such that as long as the marker 37 is visible on the proximal side of the hub 34, the clinician knows that the pump is within the tubular body 36.

The hub 34 may be provided with a first engagement structure 39 for engaging a complimentary second engagement structure on the introducer sheath to lock the insertion tool into the introducer sheath. The hub 34 may also be provided with a locking mechanism 41 for clamping onto the shaft 16 to prevent the shaft 16 from sliding proximally or distally through the insertion tool once the MCS device has been positioned at the desired location in the heart. The hub 34 may additionally be provided with a hemostasis valve to seal around the shaft 16 and also accommodate passage of the larger diameter MCS device which includes the pump. In one commercial presentation of the system, the MCS device as packaged is pre-positioned within the insertion tool and the guidewire aid is pre-loaded within the MCS device and shaft 16, as illustrated in FIG. 4.

A guidewire aid 38 (also illustrated in FIG. 8) may include a proximal opening 90 configured to slip over and removably receive the distal tip 64 and/or struts at the distal end of the inlet tube 70 that define windows of the pump inlet 66. A guidewire guide tube 83 having a lumen therethrough may be positioned within the proximal opening 90 and aligned to pass through the guidewire port 76 of the distal tip 64. The lumen of the guidewire guide tube 83 may be in communication with a distal flared funnel opening 92 which gets larger in cross-section in the distal direction. The guidewire aid 38 may be provided assembled on the MCS pump with the guidewire guide tube 83 pre-loaded along a guidewire path, for example into the MCS pump through port 76, through a portion of the fluid path within the inlet tube 70, out of the MCS pump through port 78, along the exterior of the MCS pump and back into the shaft 16 through port 80. This helps a user guide the proximal end of a guide wire into the funnel 92 through the guidewire path and into the guidewire lumen of the MCS shaft 16. A pull tab 94 may be provided on the guide wire aid 38 to facilitate grasping and removing the guidewire aid, including the guidewire guide tube 83, following loading of the guidewire. The guidewire aid 38 may have a longitudinal slit or tear line, for example along the funnel 92, proximal opening 90 and guidewire guide tube 83, to facilitate removal of the guidewire aid 38 from the MCS pump 22 and guidewire 100

Referring to FIGS. 5 and 6, an introducer kit 110 may include a guidewire 100, an introducer sheath 112, a dilator 114, and a guidewire aid 38, discussed above. The guidewire 100 may comprise an elongate flexible body 101 extending between a proximal end 102 and a distal end 104. A distal zone of the body 101 may be pre-shaped into a J tip or a pigtail, as illustrated in FIG. 6, to provide an atraumatic distal tip. A proximal zone 106 may be configured to facilitate threading through the MCS device and can extend between the proximal end 102 and a transition 108. The proximal zone 106 may have an axial length within the range of from about 100 mm to about 500 mm (e.g., about 300 mm).

The introducer kit 110 may comprise a sheath 112 and a dilator 114. The sheath 112 may comprise an elongate tubular body 116, extending between a proximal end 118 and a distal end 120. The tubular body 116 may terminate proximally in a proximal hub 122. The tubular body 116 may be expandable or may be configured to be peeled apart. The proximal hub 122 may include a proximal end port 124 in communication with a central lumen extending throughout the length of the tubular body 116 and out through a distal opening, configured for axially removably receiving the elongate dilator 114. Proximal hub 122 may additionally be provided with a side port 126, at least one and optionally two or more attachment features such as an eye 128 to facilitate suturing to the patient, and at least one and optionally a plurality of hemostasis valves for providing a seal around a variety of introduced components such as a standard 0.035″ guidewire, a 5 Fr or 6 Fr diagnostic catheter, an 0.018″ placement guidewire 100, and the insertion tool 32.

Additional details of the distal, pump region of the MCS system 10 according to some embodiments are illustrated in FIG. 7. Pump zone 60 may extend between a bend relief 62 at the distal end of shaft 16 and a distal tip 64. An inlet device 1000 may extend between a pump zone 60 and the distal tip 64. A pump inlet 66 may be in fluid communication with a pump outlet 68 by way of a flow path extending axially through an inlet tube 70, which may be part of inlet device 1000. The pump inlet may be positioned at about the transition between the inlet tube and the proximal end of distal tip 64, and in any event is generally within about 5 cm or 3 cm or less from the distal port 76. In some embodiments, the distal tip 64 is radiopaque. For example, the distal tip may be made from a polymer containing a radiopacifier such as barium sulfate, bismuth, tungsten, iodine. In some embodiments, an entirety of the MCS device is radiopaque. In some embodiments, a radiopaque marker is positioned on the inlet tube between the pump outlet 68 and the guidewire port 78 to indicate the current position of the aortic valve. Inlet tube 70 may comprise a highly flexible slotted (e.g., laser cut) metal (e.g., Nitinol) tube having a polymeric (e.g., Polyurethane) tubular layer to isolate the flow path. Inlet tube 70 may have an axial length within the range of from about 60 mm and about 100 mm and in one implementation is about 67.5 mm. The outside diameter of inlet tube 70 may typically be within the range of from about 4 mm to about 5.4 mm, and in one implementation may be about 4.66 mm. The wall thickness of inlet tube 70 may be within the range of from about 0.05 mm to about 0.15 mm. Connections between components of the MCS system 10, such as the inlet tube 70 and/or inlet device 1000, a distal tip component 64, a motor housing 74, and an impeller housing may be secured such as through the use of laser welding, adhesives, threaded or other interference fit engagement structures, via press fit, and/or via a connection device 500 as described herein.

The impeller 72 may be positioned in the flow path between the pump inlet 66 and pump outlet 68. In the illustrated embodiment, the impeller 72 is positioned adjacent to the pump outlet 68. As is discussed further below, the impeller 72 may be rotationally driven by a motor contained within motor housing 74, on the proximal side of the impeller 72. In some embodiments, the impeller 72 may be magnetically driven by a motor contained within a motor housing 74.

The MCS device may be provided in either a rapid exchange or over the wire configuration. A first guide wire port 76 is in communication, via a first guide wire lumen through the distal tip component 64 and at least a portion of the flow path in the inlet tube, with second guide wire port 78 extending through a side wall of the inlet tube 70, and distal to the impeller 72. This could be used for rapid exchange, with the guidewire extending proximally alongside the catheter from the second guidewire port 78.

The catheter may be provided in an over the wire configuration, in which the guidewire extends proximally throughout the length of the catheter through a guidewire lumen. In the over the wire embodiment of FIG. 7, however, the guidewire exits the catheter via second guidewire port 78, extends proximally across the outside of the impeller and motor housing, and reenters the catheter shaft 16 via third guidewire port 80. See also FIG. 8. The third guide wire port 80 may be located proximal to the motor, and, in the illustrated embodiment, is located on the bend relief 62. Third guide wire port 80 is in communication with a guide wire lumen which extends proximally throughout the length of the shaft 16 and exits at a proximal guidewire port carried by the proximal hub 18.

The pump may be provided assembled with a removable guidewire aid 38 having a guidewire guide tube 83 which tracks the intended path of the guidewire from the first guidewire port 76, proximally through the tip 64 and back outside of the inlet tube via second guide wire port 78 and back into the catheter via third guidewire port 80. In the illustrated implementation, the guidewire guide tube extends proximally within the catheter to a proximal end 81, in communication with, or within the guidewire lumen which extends to the proximal hub 18. The guidewire guide proximal end 81 may be positioned within about 5 mm or 10 mm of the distal end of the shaft 16, or may extend into the catheter shaft guidewire lumen for at least about 10 mm or 20 mm, such as within the range of from about 10 mm to about 50 mm. The proximal end 102 of a guidewire 100 may be inserted into the funnel 92, passing through the first (distal) guidewire port 76 and guided along the intended path by tracking inside of the guidewire guide tube. The guidewire guide tube may then be removed, leaving the guidewire in place.

In some embodiments, the distal end of the guidewire guide tube 83 is attached to the pull tab 94 of guide wire aid 38 and provided with an axially extending split line such as a weakening, slot or perforated tearable line. Removal may be accomplished such as by grasping the pull tab 94 and pulling out the guide wire tube as it splits and peels away along the split line. The inside surface of guide tube 83 may be provided with a lubricious coating, such as PTFE.

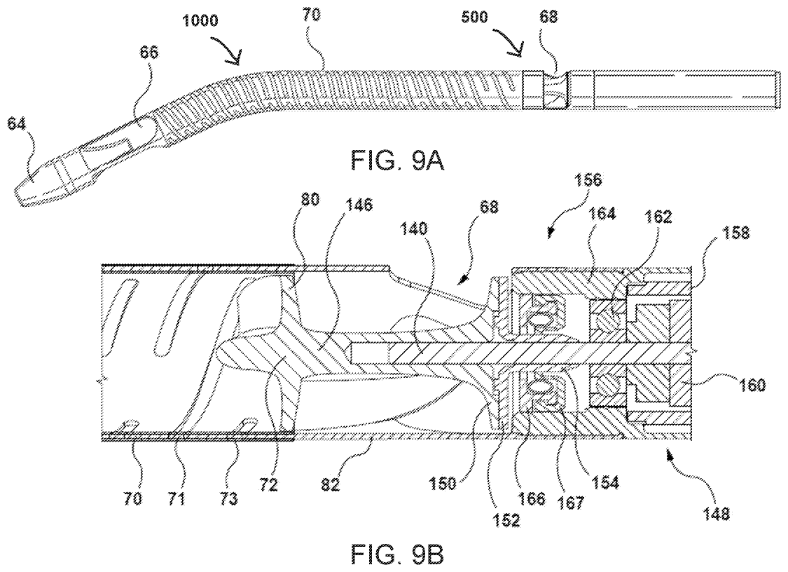

Referring to FIG. 9A, a side view is shown of an MCS device that may be used with the MCS system 10 according to some embodiments. Referring to FIG. 9B, a partial cross-sectional view is shown of a region of the MCS device of FIG. 9A, showing an embodiment of a seal, among other features. As shown in FIG. 9B, the impeller 72 may be attached to a short, rigid motor drive shaft 140. In the illustrated embodiment, the drive shaft 140 extends distally into a proximally facing central lumen in the impeller 72, such as through a proximal extension 154 on the impeller hub 146, where it may be secured by a press fit, laser weld, adhesives or other bonding technique. The impeller 72 may include a radially outwardly extending helical blade 80, which, at its maximum outside diameter, is spaced apart from the inside surface of tubular impeller housing 82 within the range of from about 40 μm to about 120 μm. Impeller housing 82 may be a proximal extension of the inlet tube 70 and/or inlet device 1000, on the proximal side of the slots 71 formed in the inlet tube 70 to provide flexibility distal to the impeller. A tubular outer membrane 73 may enclose the inlet tube and seal the slots 71 while preserving flexibility of the inlet tube. Pump outlets 68 may be formed in the sidewall of the impeller housing, axially aligned for example with a proximal portion of the impeller (e.g., a proximal 25% to 50% portion of the impeller).

The impeller 72 may comprise a medical grade titanium. This enables a CFD optimized impeller design with minimized shear stress for reduced damage of the blood cells (hemolysis) and a non-constant slope increasing the efficiency. This latter feature cannot be accomplished with a mold-based production method. Electro polishing of the surface decreases the surface roughness to minimize the impact on hemolysis.

In some embodiments, the impeller hub 146 flares radially outwardly in a proximal direction to form an impeller base 150, which may direct blood flow out of the outlets 68. A proximal surface of the impeller base 150 is secured to an impeller back 152, which may be in the form of a radially outwardly extending flange, secured to the motor shaft 140. For this purpose, the impeller back 152 may be provided with a central aperture to receive the motor shaft 140 and may be integrally formed with or bonded to a tubular sleeve 154 adapted to be bonded to the motor shaft 140. In some embodiments, the impeller back 152 is first attached to the motor shaft 140 and bonded such as through the use of an adhesive. In a second step, the impeller 72 may be advanced over the shaft and the impeller base 150 bonded to the impeller back 152 such as by laser welding.

The distal opening in the aperture in impeller back 152 may increase in diameter in a distal direction, to facilitate application of an adhesive. The proximal end of tubular sleeve 154 may decrease in outer diameter in a proximal direction to form and entrance ramp for facilitating advancing the sleeve proximally over the motor shaft and through the motor seal 156, discussed further below.

Motor 148 may include a stator 158 having conductive windings surrounding a cavity which encloses motor armature 160 which may include a plurality of magnets rotationally secured with respect to motor shaft 140. The motor shaft 140 may extend from the motor 148 through a rotational bearing 162 and also through a seal 156 before exiting the sealed motor housing 164. Seal 156 includes a seal holder 166 which supports an annular seal 167 such as a polymeric seal ring. The seal ring includes a central aperture for receiving the sleeve 154 and is biased radially inwardly against the sleeve 154 to maintain the seal ring in sliding sealing contact with the rotatable sleeve 154. The outside surface of the sleeve 154 may be provided with a smooth surface such as by electro polishing, to minimize wear on the seal.

In some embodiments, the MCS device may comprise a contactless-drive without a drive shaft 140 and seal 156, with the motor within the housing and the impeller each configured for torque transmission via magnetic coupling. The MCS device may include various features for torque transmission via magnetic coupling, for example those described in U.S. Pub. No. 2022/0161019, titled PURGELESS MECHANICAL CIRCULATORY SUPPORT SYSTEM WITH MAGNETIC DRIVE and published on May 26, 2022, the entire contents of which are incorporated by reference herein.

According to some embodiments, the MCS pump has a maximum outside diameter of less than five millimeters, in other embodiments it has an outside diameter of less than eight millimeters. According to some embodiments, the MCS pump is designed for a short-term use of less than 24 hours, in some embodiments for a use of less than ten days, in some embodiments for less than 28 days, in some embodiments for less than or equal to six months, and in some embodiments for greater than or equal to six months.

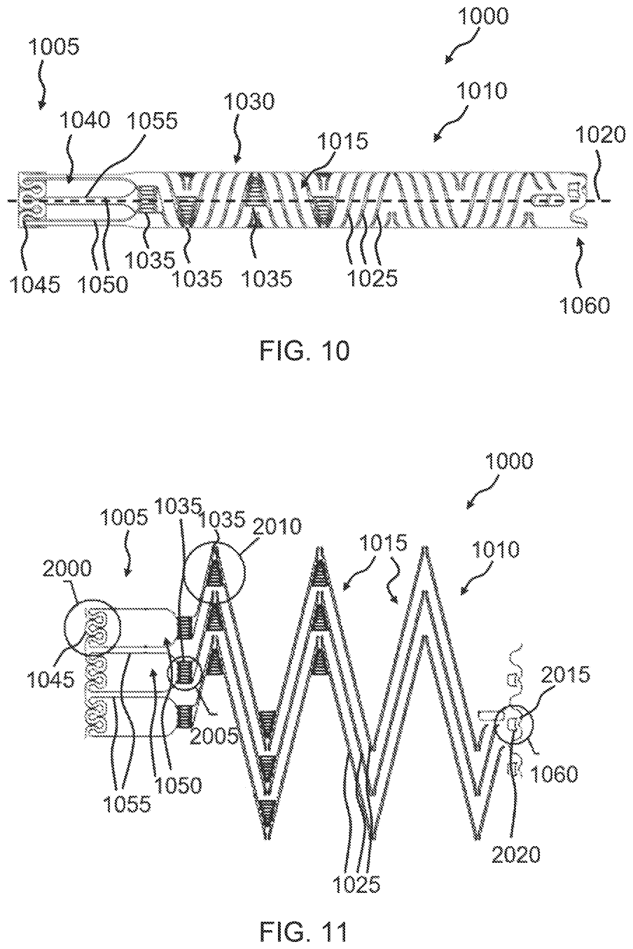

Referring to FIG. 10, a side view is shown of an inlet device 1000 according to some embodiments. Inlet device 1000 may be used in conjunction with a MCS device as an inlet to the MCS device, and/or may be used to transfer body fluid of a patient within the body. The inlet device 1000 may be used with any of the MCS systems or components described with respect to FIGS. 1-9B. Inlet device 1000 may serve generally as a tube or conduit through which, for example, the patient's blood flows. Inlet device 1000 may comprise an inlet portion 1005 and a transfer portion 1010. Inlet portion 1005 may be designed to receive body fluid into the inlet device 1000. Transfer portion 1010 may be fluidly connected to the inlet portion 1005. Transfer portion 1010 may comprise a support structure 1015. In some embodiments, the support structure 1015 may be used to support a sensor cable, a guidewire, or the like. In some embodiments, support structure 1015 may provide structural rigidity to the inlet device 1000 and prevent the inlet device 1000 from collapsing upon itself during the transfer of a body fluid and/or while under vacuum or having lower pressure internally than externally. Transfer portion 1010 may be formed to be flexible, stretchable, and/or deformable by design of the support structure 1015. In some embodiments, transfer portion 1010 may allow for the inlet device 1000 to conform, e.g. bend, twist, contort, elongate, shorten, etc., to an artery, vein, or other passageway within the body of a patient and/or accommodate movement of a patient while the inlet device 1000 is inside the patient.

In some embodiments, the inlet device 1000 may include a support structure 1015 that may comprise a plurality of structural elements 1025 that together form the support structure 1015. As shown in FIG. 10, in some embodiments the structural elements 1025 of the support structure 1015 can be arranged at an angle relative to a longitudinal axis 1020 of the inlet device 1000. Further, and according to some embodiments, the direction of the structural elements 1025 may change throughout the support structure 1015. The structural elements 1025 may be a series of zig zag struts extending longitudinally along the length of the inlet device 1000.

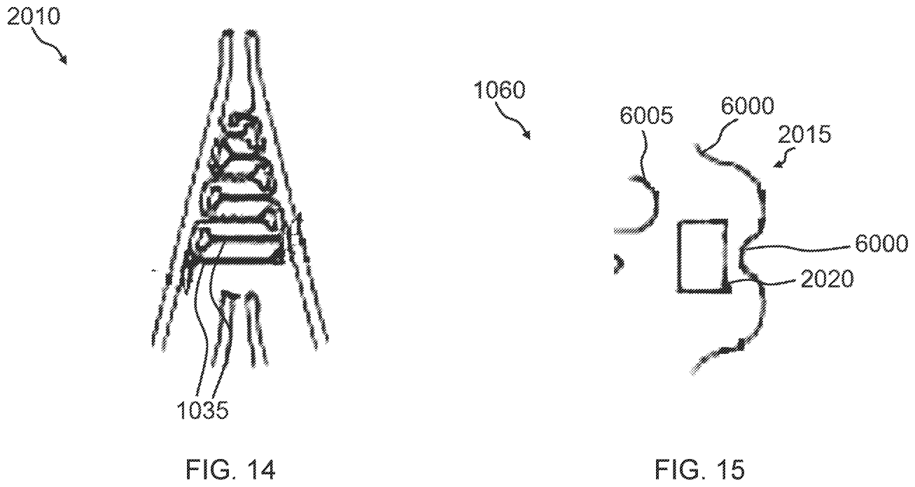

Also shown in FIG. 10, in some embodiments, the inlet device 1000 may comprise a transfer portion 1010 with a transition portion 1030 adjacent and fluidly connected to an inlet portion 1005. Transition portion 1030 may comprise a plurality of flexibility elements 1035, which can increase the deformability and/or flexibility of the transfer portion 1010 at the transition portion 1030. The flexibility elements 1035 may be substantially parallel with each other and be of about equal length or may be of variable length. In some embodiments, adjacent flexibility elements 1035 may have substantially similar lengths and form a rectangular shape in combination (shown in more detail later in FIG. 13). In some embodiments, adjacent flexibility elements 1035 may successively decrease in length and form a wedge or triangular shape in combination (shown in more detail later in FIG. 14). The flexibility elements 1035 may be structurally deformable components joining ends of adjacent structural elements 1025, which may be rigid. The flexibility elements 1035 may be polymeric, thin metal, and/or other materials. The flexibility elements 1035 may allow for adjacent structural elements 1025 to angle relative to each other in response to contortions of the overall tube away, while biasing the structural elements 1025 such that the tube is biased to revert back to a generally linear configuration.

In some embodiments, transfer portion 1010 of inlet device 1000 may comprise a cross-sectional area and/or diameter that is greater than a cross-sectional area and/or diameter of inlet portion 1005. In some embodiments, transfer portion 1010 of inlet device 1000 may comprise a cross-sectional area and/or diameter that is about 25% to about 35% greater than a cross-sectional area and/or diameter of inlet portion 1005. In some embodiments, transfer portion 1010 of inlet device 1000 may comprise a cross-sectional area and/or diameter that is about 30% greater than a cross-sectional area and/or diameter of inlet portion 1005. This increase in cross-sectional area and/or diameter may allow for a dense and even flow of body fluid through the transfer portion 1010. In some embodiments, an inner and/or outer wall and/or surface and/or spaces between structural elements 1025 of the transfer portion 1010 may be coated, covered, or filled in the case of the spaces between structural elements with a polymer material. In some embodiments, an inner wall and/or outer wall and/or surface of the transfer portion 1010 may comprise a polymeric sleeve. A polymer coating and/or polymeric sleeve may prevent, for example, components of body fluid from depositing on the inner wall and/or surface of the transfer portion 1010 and thus reduce and/or prevent clogging of the inlet device 1000, which could have life-threatening consequences for the patient.

As shown in FIG. 10, in some embodiments the support structure 1015 is formed at least partially wound. Also shown in FIG. 10, in some embodiments inlet portion 1005 comprises a second support structure 1040 that is connected to support structure 1015. In some embodiments, second support structure 1040 is wound with support structure 1015 to form the inlet device 1000. In some embodiments, the second support structure 1040 comprises, at its end, windings 1045. Windings 1045 may be formed in a continuous S-like shape with a variable number of bends. In some embodiments, second support structure 1040 may comprise a plurality of bars 1055 that span from the transfer portion 1010 across a portion of the inlet portion 1005 and form openings 1050. In some embodiments, second support structure 1040 may comprise a plurality of bars 1055 that span from the transition portion 1030 of the transfer portion 1010 across a portion of the inlet portion 1005 and form openings 1050. In some embodiments, and as shown in FIG. 10, bars 1055 of the second support structure 1040 may extend longitudinally with longitudinal axis 1020 of the inlet device 1000. In some embodiments, and as shown in FIG. 10, openings 1050 may also extend longitudinally with longitudinal axis 1020 of the inlet device 1000. In some embodiments, bars 1055 may extend in a curvilinear path relative to longitudinal axis 1020 of the inlet device 1000, creating openings 1050 that may also extend in a curvilinear path. Body fluid may enter openings 1050 and then pass through transfer portion 1010 of the inlet device 1000. In some embodiments, a framework is formed by the bars 1055 of the second support structure 1040 that may ensure a flow of body fluid through inlet device 1000 is maintained, for example, by preventing a blood vessel from contracting and thus disturbing flow. In some embodiments with curvilinear extending bars 1055 and openings 1050, such curvilinear arrangement may introduce a rotation of body fluid into the inlet device 1000 and aid in the flow of the body fluid.

As shown in FIG. 10, in some embodiments transfer portion 1010 comprises a connection region 1060 at its end opposite of inlet portion 1005. Inlet device 1000 may be connected to other components and/or systems at connection region 1060, for example to the MCS systems described herein. Further details of a connection region 1060 according to some embodiments are described herein, for example with respect to FIG. 15. In some embodiments, connection region 1060 may comprise a connection device or an element thereof as described herein.

In some embodiments, support structure 1015 and/or second support structure 1040 of the inlet device 1000 are comprised of a super-elastic material, for example Nitinol. In some embodiments, inlet device 1000 is flexible, deformable, and/or stretchable and can withstand forces, loads and stresses associated with insertion and navigation in vivo. In some embodiments, inlet device can bend about 210° and remain patent/open for the flow and/or transfer of body fluid.

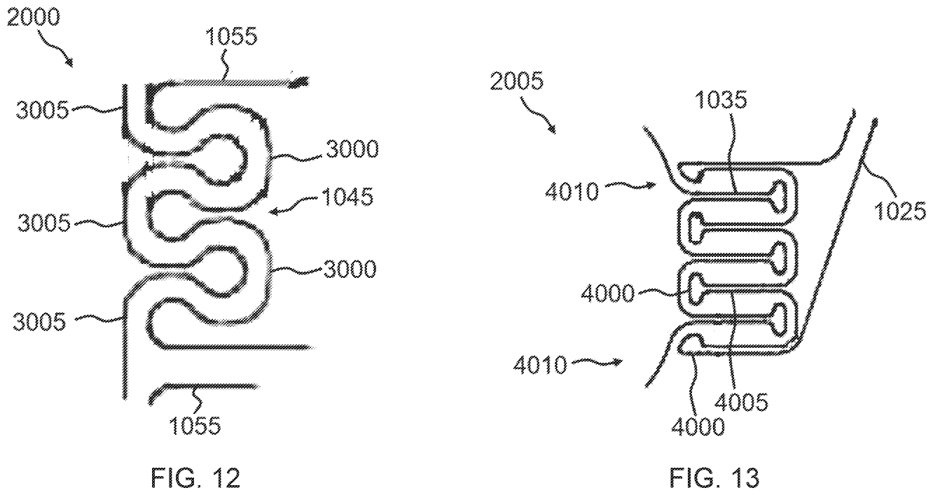

Referring to FIG. 11, a schematic view is shown of the inlet device 1000 shown in FIG. 10 in an unwound and/or unrolled state according to some embodiments. Also shown in FIG. 11 are magnified areas with corresponding detail views in other figures, specifically magnification of a winding region 2000 for reference in FIG. 12, magnification of a first engagement region 2005 for reference in FIG. 13, magnification of a second engagement region 2010 for reference in FIG. 14, and magnification of a connection region 1060 for reference in FIG. 15.

As shown in FIG. 11 and according to some embodiments, winding region 2000 may be located at an end of the inlet portion 1005 opposite transfer portion 1010. Winding region 2000 may comprise windings 1045, and as discussed relative to FIG. 10, the windings 1045 may be formed in a continuous S-like shape with a variable number of bends. As shown, windings 1045 may be adjacent to at least one of the openings 1050 of the inlet portion 1005. According to the embodiment shown, inlet device 1000 may comprise three similarly shaped openings 1050 formed by bars 1055.

As shown in FIG. 11 and according to some embodiments, at a first engagement region 2005 flexibility elements 1035 connect to bars 1055 opposite of windings 1045. As shown, flexibility elements 1035 at first engagement region 2005 may be formed substantially parallel with each other and be of about equal length. Further as shown, flexibility elements 1035 at first engagement region 2005 may be designed to create a transition between the inlet portion 1005 and transfer portion 1010. In some embodiments, structural elements 1025 of support structure 1015 may connect to flexibility elements 1035 at first engagement region 2005 opposite of bars 1055.

As shown in FIG. 11 and according to some embodiments, at a second engagement region 2010 flexibility elements 1035 may connect ends of structural elements 1025 of support structure 1015. As shown, flexibility elements 1035 at second engagement region 2010 may be formed substantially parallel with each other and be of variable length so as to form a triangular shape in combination at the connection of structural elements 1025. Further as shown, flexibility elements 1035 at second engagement region 2010 may be designed to encourage flexibility of inlet device 1000.

As shown in FIG. 11, in some embodiments the support structure 1015 of transfer portion 1010 may be comprised of a serrated and/or zig-zagged material layer with one or more structural elements 1025. Structural elements 1025 may connect to each other through a flexibility element 1035 or may be of a continuous serrated and/or zig-zagged shape.

As shown in FIG. 11 and mentioned relative to FIG. 10, in some embodiments transfer portion 1010 comprises a connection region 1060 at the end opposite of the inlet portion 1005. Inlet device 1000 may be connected to other components and/or systems at connection region 1060, for example to an MCS system. As shown, the inlet device 1000 at connection region 1060 may comprise a corrugated and/or a wave-like edge 2015 facing away from inlet portion 1005, which may be of substantially similar or of variable depth undulations. Furthermore, in some embodiments connection region 1060 may comprise one or more through openings 2020. Further details of a connection region 1060 according to some embodiments will be shown later in FIG. 15. In some embodiments, connection region 1060 may comprise a connection device or an element thereof as described herein.

Referring to FIG. 12, a detailed view is shown of the winding region 2000 of the inlet device 1000 shown also in FIG. 10 and FIG. 11 and according to some embodiments. As shown in this magnified view, the winding region 2000 may comprise windings 1045 that may be formed in a continuous S-like shape with a variable number of bends. Windings 1045 may fulfill a spring-like function against external forces. As shown, in some embodiments winding region 2000 may comprise windings 1045 with two complete turns 3000 and three partial turns 3005. Further as shown, in some embodiments a partial turn 3005 of windings 1045 may connect to a bar 1055 as described herein. The windings 1045 may be Nitinol or other materials.

Referring to FIG. 13, a detailed view is shown of the first engagement region 2005 of the inlet device 1000 shown also in FIG. 10 and FIG. 11 and according to some embodiments. As shown in this magnified view, first engagement region 2005 may comprise interwoven flexibility elements 1035 substantially parallel to each other and of about equal length, each with an end 4000 and neck 4005 and forming a substantially T-shape. As shown, the end 4000 of the flexibility element 1035 may be formed wider than the neck 4005. Further, in some embodiments the first engagement region 2005 may further comprise edge regions 4010 wherein engagement sections 1035 may connect to bars 1055 of the inlet portion 1005 (bars 1055 not shown). In some embodiments and as shown, flexibility elements 1035 at edge regions 4010 may be substantially P-shaped (not T-shaped). In some embodiments, flexibility elements may be arranged coming from opposite directions of the inlet device 1000. In some embodiments, a clearance between adjacent flexibility elements 1035 is created and maintained around the flexibility elements. In some embodiments, the clearance between adjacent flexibility elements 1035 is arranged in a serpentine-like fashion and evenly around the flexibility elements 1035. In some embodiments and as shown in FIG. 13, the first engagement section may be designed as a transition between the inlet portion 1005 and the transfer portion 1010, with bars 1055 of the inlet portion 1005 connecting at one end and structural elements 1025 of the transfer portion 1010 connecting at the other end.

Referring to FIG. 14, a detailed view is shown of the second engagement region 2010 of the inlet device 1000 shown also in FIG. 10 and FIG. 11 and according to some embodiments. As shown in this magnified view, second engagement region 2010 may comprise a plurality of substantially parallel flexibility elements 1035 similar to the first engagement region 2005 described in FIG. 13, but in this region flexibility elements 1035 may be of variable length. As shown in FIG. 14, adjacent flexibility elements 1035 may successively decrease in length and form a wedge or triangular shape in combination.

Referring to FIG. 15, a detailed view is shown of the connection region 1060 of the inlet device 1000 shown also in FIG. 10 and FIG. 11 and according to some embodiments. As shown, the inlet device 1000 at connection region 1060 may comprise a corrugated and/or a wave-like edge 2015 facing away from inlet portion 1005. As shown, edge 2015 of connection region 1060 may comprise a plurality of inward and outward curvatures 6000, of similar or of different depths. In some embodiments, curvatures 6000 of different depth may create an edge 2015 that is undulating and with a variable profile. In some embodiments, a through-opening 2020 may be located adjacent to where edge 2015 comprises a less deep curvature 6000. The opening 2020 may be polygonal in shape, for example rectangular as shown in FIG. 15, or it may be round, oval, or with rounded corners. In some embodiments, connection region 1060 may comprise at least one further opening 6005, which as shown in FIG. 15 is rounded. Further, the opening 6005 may be polygonal in shape, for example rectangular, or it may be round, oval or with rounded corners. Inlet device 1000 may be connected to other components and/or systems at connection region 1060, for example to an MCS system. In some embodiments, inlet device 1000 may be connected to other components and/or systems at connection region 1060 by way of complementary features to edge 2015, one or more through openings 2020, one or more further openings 6005, or any combination thereof. In some embodiments, connection region 1060 may comprise a connection device or an element thereof as described herein.

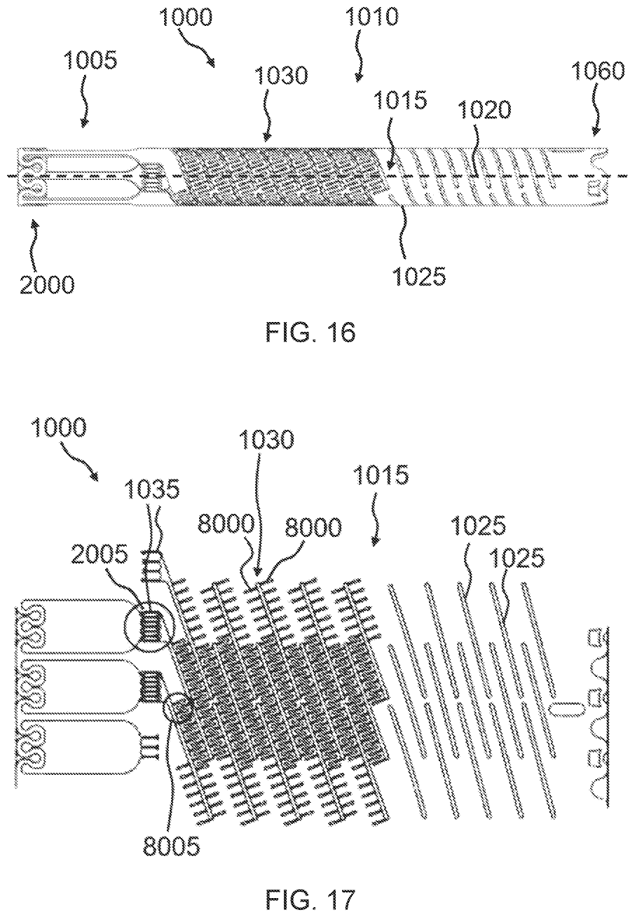

Referring to FIG. 16, a side view is shown of the inlet device 1000 according to some embodiments. With the exception of support structure 1015, the inlet device 1000 shown in FIG. 16 may comprise features and/or elements that correspond to features and/or elements of embodiments described with respect to FIGS. 10, 12, 13, and 15. As shown in FIG. 16, in some embodiments the support structure 1015 may comprise a plurality of structural elements 1025. In some embodiments, structural elements 1025 may be arranged at an angle to the longitudinal axis 1020 of the inlet device 1000. Also shown in FIG. 16, in some embodiments inlet device 1000 may comprise a transition portion 1030, which may contribute to the flexibility of the inlet device 1000.

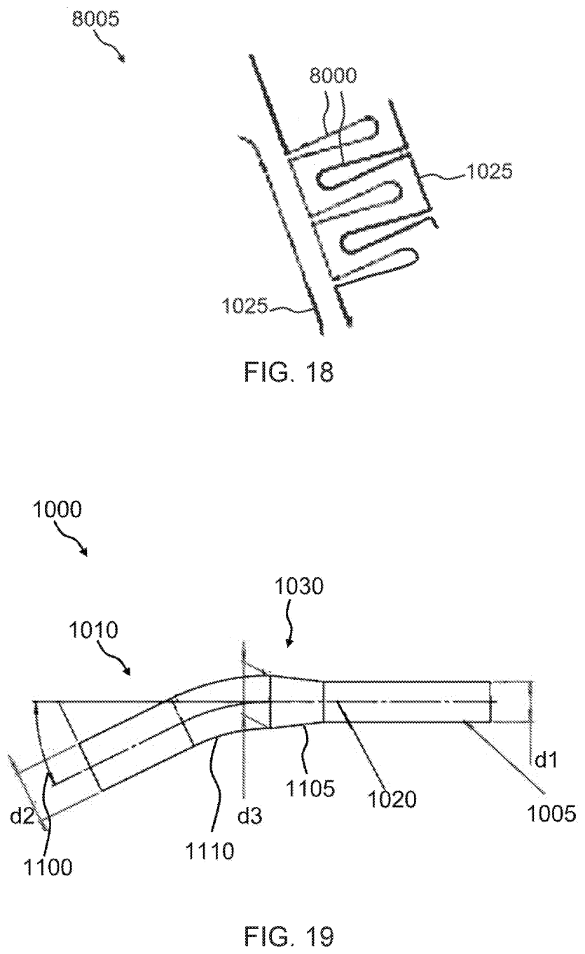

Referring to FIG. 17, a detailed view is shown of the inlet device 1000 shown in FIG. 16 in an unwound and/or unrolled state according to some embodiments. Also shown in FIG. 17 are magnified detail areas, specifically magnification of the first engagement region 2005 as described previously in FIG. 13, and magnification of a finger engagement region 8005 for reference in FIG. 18. As shown, in some embodiments structural elements 1025 at transition portion 1030 may comprise finger elements 8000, which at adjacent structural elements may face and be arranged offset from each other. Further details of the finger engagement region 8005 according to some embodiments are shown in FIG. 18.

Referring to FIG. 18, a detailed view is shown of the finger engagement region 8005 of the inlet device 1000 according to some embodiments. As shown, structural elements 1025 located at transition portion 1030 may comprise a plurality of interwoven finger elements 8000. Further, in some embodiments the finger elements 8000 of adjacent structural elements 1025 may be arranged offset to each other and facing each other. In some embodiments, the finger elements 8000 of adjacent structural elements 1025 may be arranged offset to each other, may face each other, and may integrate with one another in such a way as to create a tight spacing between each other yet still maintaining a void space between adjacent finger elements 8000. Finger elements 8000 may be of substantially similar length and arranged parallel to each other. Finger elements 8000 may be approximately drop-shaped, as shown in FIG. 18, with an end that connects to structural element 1025 being tapered in relation to a free end that faces away from the structural element 1025 it connects to. Finger elements 8000 may alternatively be substantially linearly arranged with a full bend at a free end that is opposite the corresponding structural element 1025 to which the finger element connects.

Referring to FIG. 19, a side view is shown of the inlet device 1000 according to some embodiments comprising the inlet portion 1005 and transfer portion 1010 as previously described. The inlet device 1000 in FIG. 19 may include any of the features described herein with respect to FIGS. 1-18. According to the example shown in FIG. 19 and to some embodiments, the inlet device 1000 may be deformable and can be bent at an angle 1100. As shown and in some embodiments, inlet portion 1005 may have a diameter d1 that is smaller than a diameter d2 of transfer portion 1010. Further as shown and in some embodiments, a transition portion 1030 may be located between and in fluid communication with the inlet portion 1005 and the transfer portion 1010, and may be comprised of a transition 1105 wherein the diameter of the inlet device 1000 changes from d1 of the inlet portion 1005 to d2 of the transfer portion. In some embodiments, transition portion 1030 has a diameter d3 that matches diameter d2 of the transfer portion 1010 where it connects to the transfer portion 1010, and a diameter that matches diameter d1 of the inlet portion 1005 where it connects to the inlet portion 1005. In some embodiments, the diameter of an inlet device 1000 increases across the transition portion 1030. As shown in FIG. 19, in some embodiments the inlet portion 1005, the transition portion 1030, and the transfer portion 1010 share a common longitudinal axis. In some embodiments, the inlet device 1000 may be flexible and bend at any location along its longitudinal length.

In some embodiments, an inlet device 1000 as described herein may be formed from sheet metal and deformed and/or rolled into final shape. In some embodiments, an inlet device 1000 as described herein may be formed from a tube and/or tubular segments.

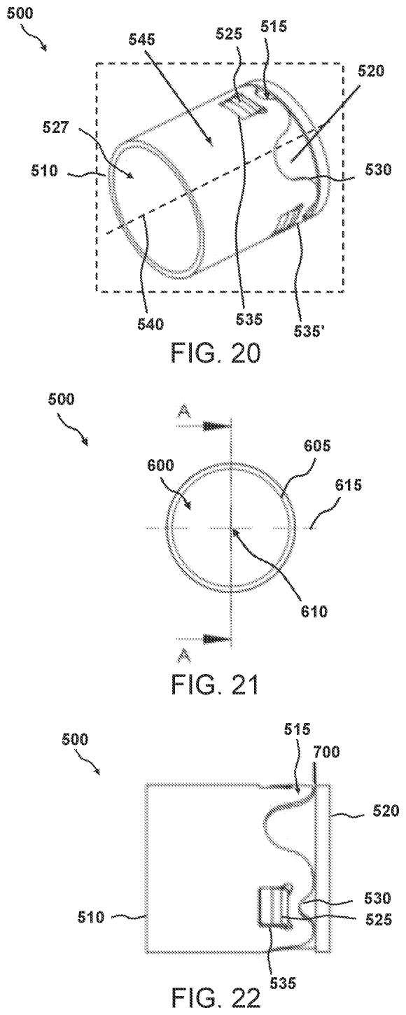

Referencing FIG. 20, a connecting device 500 according to some embodiments may comprise a receiving element 510 and an insertion element 520 as shown connected together. The connecting device 500 may be used with any of the various embodiments of the MCS system and related components as shown and described with respect to FIGS. 1-19. The connecting device 500 may be designed to allow components of an MCS system to releasably attach and detach. In some embodiments, connecting device 500 may be designed to allow components of an MCS system to readily attach but not readily detach. Further, the connecting device 500 may be designed to allow for the transmission of acting forces. In the connected/attached state, receiving element 510 and insertion element 520 may form an internal cavity 527 along a shared longitudinal axis 540, which can allow for the passage of control cabling, guidewires, fluids, and the like. In some embodiments, internal cavity 527 may house an impeller 72 of the MCS device.

The receiving element 510 may be formed in a substantially cylindrical shape, such as a tube, with an outer surface and an inner surface. The receiving element 510 may comprise a receiving structure 515 at an end that faces insertion element 520. The receiving structure 515 may comprise one or more through-openings 535 and 535′ on the lateral surface 545 of the receiving element 510 as shown in FIG. 20 that form an opening between the inner and outer surfaces of the receiving structure 515. The through-openings 535 and/or 535′ may be configured to receive a corresponding slide-on ramp 525 of the insertion element 520 as described further below. The one or more through-openings 535 and 535′ may be polygonal in shape. Alternatively, the one or more through-openings 535 and 535′ may be round or with rounded corners. The receiving element 510 may comprise a wave-like longitudinal edge 530 which faces insertion element 520 as shown in FIG. 20. The wave-like longitudinal edge 530 may be sinusoidal-like with regular or irregular peaks and valleys.

The insertion element 520 may be formed in a substantially cylindrical shape, such as a tube, with an outer surface and an inner surface. The inner surface of the insertion element 520 may be substantially similar to the inner surface of the receiving element 510, such that they have substantially similar inner diameters. The insertion element 520 may comprise at least one of the slide-on ramps 525. The slide-on ramp 525 may be configured to slide inside an inner surface of the receiving structure 515 and lock into the through-opening 535 or 535′ of the receiving structure in a self-locking manner. To enable a slide-on ramp 525 to slide inside an inner surface of the receiving structure 515, the slide-on ramp 525 of the insertion element 520 may deform towards longitudinal axis 540 until it is located within a through-opening 535 or 535′ of the receiving structure 515, and then self-expand outward to lock into place. Alternatively or in combination, the receiving structure 515 may deform away from longitudinal axis 540 to allow a slide-on ramp 525 to slide inside its inner surface until a slide-on ramp 525 is located within a through-opening 535 or 535′. The insertion element 520 may comprise a raised wave-like feature on its outer surface complementary to the wave-like longitudinal edge of the receiving structure. This raised wave-like feature may aid in joining and/or aligning the insertion element 520 to the receiving element 510 together through their complementary shapes. In some embodiments, this raised wave-like feature may aid in joining and/or aligning the insertion element 520 to the receiving element 510 together through their complementary shapes and allow for only one attached orientation. Further, such complementary joining of these features, when embodied, may aid in force transmittal between the receiving element 510 and the insertion element 520.

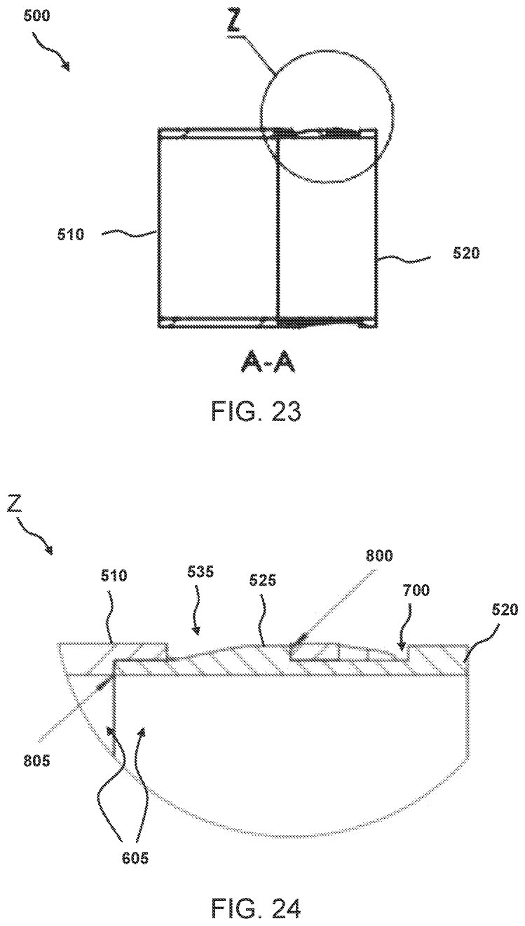

Referring to FIG. 21, a front end view is shown of a connecting device 500 according to FIG. 20 and to some embodiments. As shown, in some embodiments the connecting device 500 comprises a circular cross section with a round inner cavity 600 and a wall 605. A cutting axis A-A is shown, and the resulting cross-sectional view is shown in FIG. 23. The longitudinal axis 540 of FIG. 20 extends, according to this embodiment, through an intersection 610 of the cutting axis A-A with a transverse axis 615 perpendicular to the cutting axis A-A.

Referring to FIG. 22, a side view is shown of the connecting device 500 according to FIG. 20 and to some embodiments with the receiving element 510 and the insertion element 520 connected/attached. As depicted, in some embodiments the wave-like longitudinal edge 530 of the receiving structure 515 may comprise waves that vary in their depth along the longitudinal axis of the receiving structure 515. Also shown, and in some embodiments of connecting device 500, is an expansion joint 700 located between the receiving structure 515 of the receiving element 510 and the insertion element 520. In some embodiments and as shown, the expansion joint 700 is defined by the relative arrangement of the through-opening 535 of the receiving structure 515 and the slide-on ramp 525 of the insertion element 520 when locked into the through-opening 535. The expansion joint 700 may compensate for tolerances in manufacturing.

Referring to FIG. 23, a cross-section view is shown of the connecting device 500 as taken along the line A-A in FIG. 21. The connecting device 500 may include any features of the embodiments of FIGS. 20 through 22 and some embodiments with the receiving element 510 and the insertion element 520 connected/attached. According to this embodiment, the receiving element 510 and the insertion element 520 may have substantially similar inner surfaces that form a smooth transition where the elements meet. In some embodiments, the receiving element 510 and the insertion element 520 may have substantially similar inner diameters so that a smooth transition is formed where the elements meet. In embodiments where there is a smooth inner surface transition between receiving element 510 and insertion element 520, the smooth inner surface transition may enable trouble-free passage of any cabling, fluids, etc. (for example, no step or transition that a cabling can snag or catch on). Detail callout Z is shown in FIG. 24.

Referring to FIG. 24, a detailed cross-sectional view is shown of a connecting device 500 as taken from detail Z in FIG. 23. Shown specifically in FIG. 24 is a cross-section of an embodiment of how the slide-on ramp 525 of the insertion element 520 may interact with the through-opening 535 of the receiving element 510. As shown, in some embodiments, the slide-on ramp 525 comprises a stop 800 at one end, which may engage with through-opening 535 in the assembled state. In some embodiments, an engagement between a stop 800 at one end of a slide-on ramp 525 of an insertion element 520 and a through-opening 535 of a receiving element 510 may prevent the insertion element 520 and the receiving element 510 from coming apart and/or separating. In some embodiments, an engagement between a stop 800 at one end of a slide-on ramp 525 of an insertion element 520 and a through-opening 535 of a receiving element 510 may prevent the insertion element 520 and the receiving element 510 from coming apart and/or separating unless an inward radial force is applied to the slide-on ramp 525. As shown, in some embodiments a slide-on ramp 525 may comprise a stop 800 at one end and a reduced thickness at its opposite end, so as to form a ramp that may facilitate insertion of the insertion element 520 into a receiving element 510 but not easy removal. As shown, in some embodiments receiving element 510 may comprise a step-like recess 805 at its inner surface which can engage one end of the insertion element 520 when connected. In some embodiments receiving element 510 may comprise a step-like recess 805 at its inner surface which can engage one end of the insertion element 520 when connected to form a smooth transition of the inner surfaces of the receiving element 510 and insertion element 520. In some embodiments, the features as shown in FIG. 24 produce a connecting device 500 that is substantially free of play and slipping.

In some embodiments, the receiving element 510 and/or insertion element may be comprised of a superelastic material (for example, Nitinol). In some embodiments the receiving element 510 and/or insertion element may be comprised of titanium. In some embodiments the receiving element 510 and/or insertion element may be comprised of a superelastic material (for example, Nitinol) and/or titanium. In some embodiments, the receiving element 510 and/or insertion element may be comprised of materials that are body-safe or biocompatible. In some embodiments, the receiving element 510 and/or insertion element may be comprised of materials that allow for a form-fit and frictional connection with minimal wall thickness.

In some embodiments, a method for joining and/or assembling a connecting device 500 comprises providing a receiving element 510 with a receiving structure 515 and an insertion element 520 with at least one slide-on ramp 525, and pushing and/or inserting the insertion element 520 into the receiving structure 515 of the receiving element 510, wherein the slide-on ramp 525 of the insertion element 520 connects to the receiving structure 515 in a form-fitting, non-positive, force-locking, and/or self-locking manner. In some embodiments, a method for joining and/or assembling a connecting device 500 comprises providing a receiving element 510 with a receiving structure 515 and an insertion element 520 with at least one slide-on ramp 525, and pushing and/or sliding the receiving structure 515 of receiving element 510 over the insertion element 520, wherein the slide-on ramp 525 of the insertion element 520 connects to the receiving structure 515 in a form-fitting, non-positive, force-locking, and/or self-locking manner. In some embodiments, the connecting of a slide-on ramp 525 of the insertion element to a receiving structure 515 of the receiving element 510 comprises locating the slide-on ramp 525 within a through-opening 535 of the receiving structure 515. In some embodiments, a method for joining and/or assembling a connecting device 500 comprises cooling a Nitinol component of the connecting device, such as the receiving element 510, to a temperature below the Nitinol's austenite transformation temperature prior to pushing and/or inserting the insertion element 520 into the receiving element 510.

In some embodiments, a method for disassembling and/or unjoining a connecting device 500 comprises providing a receiving element 510 with a receiving structure 515 and an insertion element 520 with at least one slide-on ramp 525 in a connected and/or assembled configuration wherein the at least one slide-on ramp 525 connects to the receiving structure in a form-fitting, non-positive, force-locking, and/or self-locking manner, applying an inward radial force to the at least one slide-on ramp 525, and pulling apart the receiving element 510 and the insertion element 520. In some embodiments, a method for disassembling and/or unjoining a connecting device 500 comprises cooling a Nitinol component of the connecting device, such as the receiving element 510, to a temperature below the Nitinol's austenite transformation temperature prior to pulling apart the receiving element 510 and the insertion element 520.

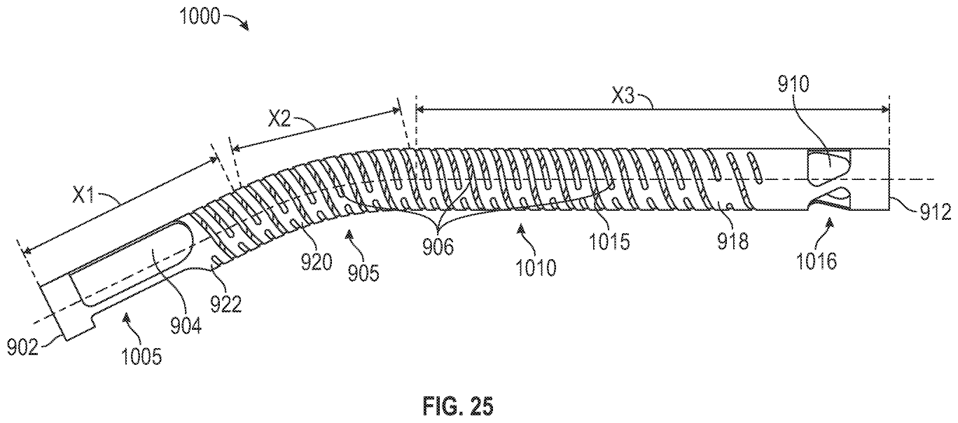

Referring to FIG. 25, a side view is shown of the inlet device 1000 according to some embodiments. As shown, the inlet portion 1005 can include one or more inlet openings 904. In the embodiment of FIG. 25, the inlet device 1000 can additionally include an outlet portion 1016 having and one or more outlet openings 910. The inlet device 1000 may have multiple sections. For example, a proximal section 918. The proximal section 918 can extend from a proximal end 912 of the inlet device 1000 to a proximal end of a middle section 920. The middle section 920 can extend from a distal end of the proximal section 918 to a proximal end of a distal section 922. The distal section 922 can extend from a distal end of the middle section 920 to a distal end 902 of the inlet device 1000. The proximal section 918 can be straight or generally straight in an unconstrained state. In some embodiments, the proximal section 918 can have a length X3. The length X3 can be about 37 mm in length, or may be in a range of about 30 mm to 50 mm. The middle section 920 can have a length X2. The middle section 920 may have a bend or curve over the length X2 in an unconstrained state. The length X2 may be about 13.61 mm in length, or may be in a range of about 10 mm to 40 mm. The distal section 922 may be straight or generally straight. The distal section 922 may have a length X1. The length X1 may be about 16.89 mm in length or may be in a range of about 10 mm to 25 mm (e.g., in a range of 16 mm to 18 mm).

In some embodiments, the support structure 1015 may include a plurality of structural elements and/or flexibility elements that together form the support structure 1015. In some embodiments, the support structure 1015 can include a cut pattern 905. The cut pattern 905 may reduce the stiffness of the inlet device 1000 or portions thereof. The cut pattern 905 can provide a desired flexibility of the inlet device 1000 or portions thereof. Different cut patterns 905 may result in different flexibility profiles.

In some embodiments, a specific pattern 905 may be selected that provides a desired flexibility profile for the minimally invasive insertion of the device 1000 through the femoral artery.

The cut pattern 905 may include a series of features 906 formed in the inlet device material. The features 906 may be cuts, windows, bars, slits or other shapes cut into the inlet device material. The shape of the individual features, size of the features, spacing between the features, and/or angles of the features may vary to offer changes in flexibility over the length of the inlet device 1000. For example, in some embodiments, the features 906 may be shaped, sized, spaced apart, oriented, or otherwise configured so that the inlet device 1000 is more flexible at and/or near the distal end 902 than at or near the proximal end 912. In some embodiments, the features 906 may be shaped, sized, spaced apart, oriented, or otherwise configured so that the flexibility of the inlet device 1000 increases from the proximal end 912 to the distal end 902. For example, the flexibility of the inlet device 1000 may increase gradually, along the length of the inlet device or at least along a portion of the length of the inlet device such as the proximal section 918. The gradual increase in flexibility may be proportional or exponential with respect to the distance from the proximal end 912. In some embodiments, the features 906 may be shaped, sized, spaced apart, oriented, or otherwise configured so that a gradual bending behavior can be achieved over the entire length of the inlet device, or at least over a portion of the length such as the proximal section 918, for example, so that the inlet device 1000 does not kink at the proximal end 912 under distal loading. In some embodiments, the bar width (e.g., the distance between adjacent features 906) decreases toward the distal end 902 (e.g., from at or near the proximal end 912 to at or near the distal end 902). In this way, in some embodiments, a gradual bending behavior can be achieved over a length (e.g., the entire length, at least a portion of the length, the length of the proximal section 918) of the inlet device, for example, so that the inlet device 1000 does not kink at the proximal end 912 under distal loading.

In some embodiments, the cut pattern 905 may be added to the body of the inlet device 1000 by a suitable method such as laser cutting or water jet cutting. In some embodiments, the inlet device 1000 may be formed of a superelastic material (for example, Nitinol), metal (for example, Titanium Grade V), and/or stainless steel.

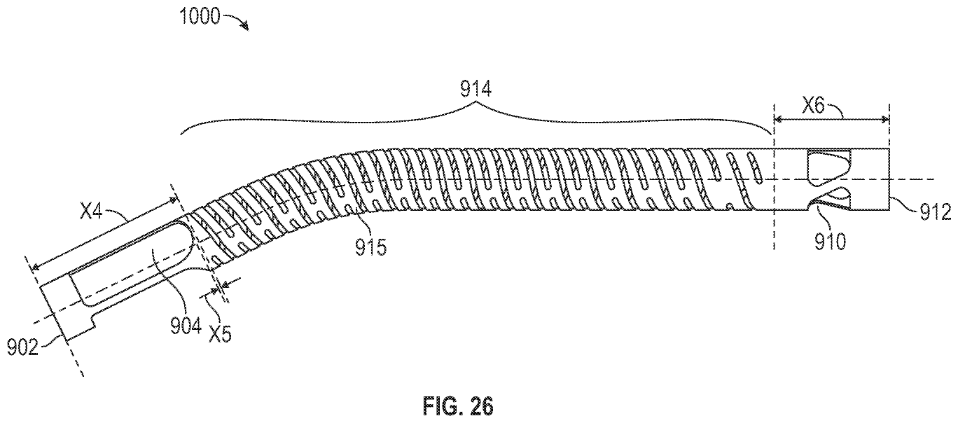

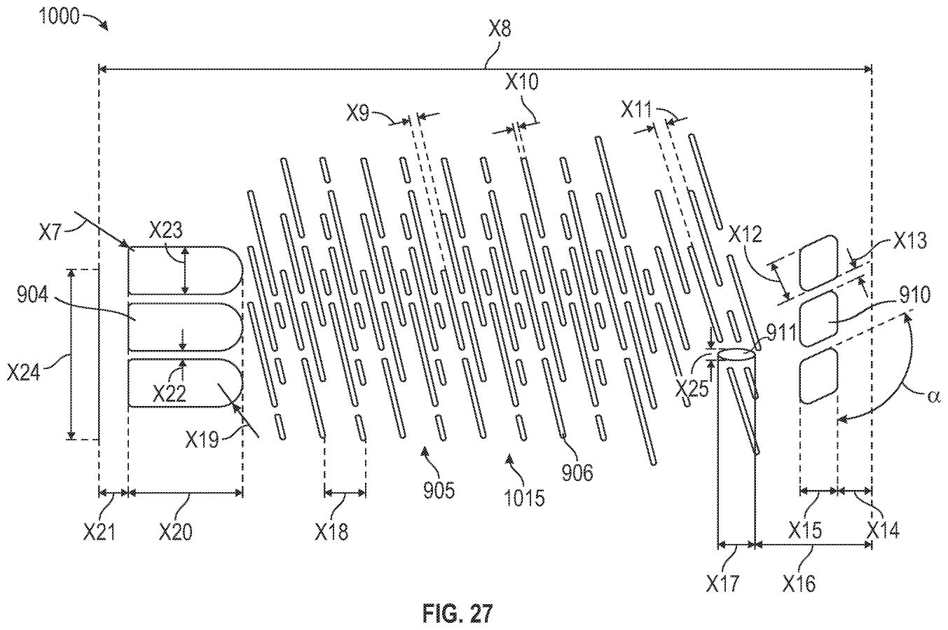

Referring to FIG. 26, a side view is shown of the inlet device 1000 of FIG. 25 with a flexible coating or covering 915 applied according to some embodiments. The coating 915 may be added to the inlet device 1000 to close the features of the previously described cut pattern 905. The coating 915 can help maintain flexibility of the inlet device 1000 and close openings for guiding flow within. In some embodiments, the inlet device 1000 may have a coated area 914 between the inlet openings 904 and outlet openings 910. For example, a proximal end of the coated area 914 may be positioned a length X6 from the proximal end 912 of the inlet device 1000. The length X6 can be about 8.9 mm or in a range of 4 mm to 15 mm. A distal end of the coated area 914 may be positioned at a length X4 from the distal end 902. The length X4 may be about 12.7 mm or in a range of 10 mm to 20 mm. In another embodiment the flexible coating or covering 915 may extend to the proximal end 912 and/or to the distal end 902 except for the openings 910, 904. In some embodiments the distance X5 between the distal end of the coating 914 to the proximal edge of the inlet opening 904 may be in a range of about 0 mm to about 0.5 mm. In some embodiments, the distance X5 is in the space between the most distal cut 906 and the inlet opening 904.

In some embodiments, the coating can be formed of polyurethane, for example, but other materials such as silicone, polyethylene (PE) or polytetrafluoroethylene (PTFE) are also conceivable.

Referring to FIG. 27, a detailed view is shown of the inlet device 1000 shown in FIG. 25 in an unwound and/or unrolled state according to some embodiments. Also shown in FIG. 25 are magnified detail areas. The inlet device 1000 may have various dimensions as shown in millimeters. The following dimensions are just one embodiment and some or all dimensions may be different in other embodiments. The inlet device 1000 may have an overall length X8 of about 67.5 mm, or in a range of 60 mm to 90 mm. The inlet device 1000 may have a circumference when rolled or assembled X24 of about 14.765 mm. The inlet openings 904 may be a distance X21 from the distal end 902 of about 2.5+/−0.05 mm, or in a range of about 2 mm to 11 mm. There may be a plurality of inlet openings 904 that are identical in size. In some embodiments, the inlet openings 904 may have a length X20 of about 10 mm, or in a range of about 6 mm to 12 mm. The inlet openings 904 may have a width X23 of about 4.12 mm, or in a range of about 3 mm to 6 mm. The inlet openings 904 may have one or more corners with a radius X7 of about 0.5 mm. The inlet openings 904 may have a radius X19 of about 2 mm, or approximately half the width of X23. The inlet openings 904 may have a space between X22 of about 0.8 mm, or in a range of about 0.6 mm to 2 mm (e.g., in a range of 1.2 mm to 1.5 mm).

In some embodiments, the outlet openings 910 may be a distance, X14, from the proximal end 912 of about 3.0+/−0.05 mm, or in a range of 3 mm to 4.5 mm. The outlet openings 910 may be all identical in size. There may be a plurality (e.g., 3, 4, 5, 6, 7, 8) of outlet openings 910 that are identical in size. In some embodiments, the outlet openings 910 may have a length X15 of about 3.30 mm, or in a range of 2.5 mm to 4 mm. The outlet openings 910 may have a width X12 of about 3.6 mm, or about 1.53 mm, or in a range of 1.5 mm to 3.8 mm. The outlet openings 910 may have a space between X13 of about 0.86 mm, or 0.55 mm, or in a range of about 0.5 mm to 1 mm. The outlet openings 910 may have a generally rectangular or rhomboid shape, optionally with radiused corners. Each of the outlet openings 910 may have a first and second opposing parallel edges that are perpendicular to the longitudinal axis and a third and fourth opposing parallel edges that are at an angle α to the first and second edges, wherein the angle α may be 90 degrees plus or minus 25 degrees.

In some embodiments, there may be a guidewire port 911 in the inlet device 1000. The guidewire port 911 may be oval, circular, or oblong. The guidewire may be positioned into the inlet tube 1000 from the distal end and passed back out through the guidewire port 911 so that the guidewire can bypass the impeller. The guidewire can run alongside the MCS catheter or back into a guidewire lumen in the MCS catheter. In embodiments with an oblong shape, the guidewire port 911 may have a length X17 of about 3.2 mm or in a range of 2.5 mm to 4 mm. The guidewire port 911 may have a width X25 of about 1 mm, or in a range of about 0.80 mm to 1.5 mm. The guidewire port 911 may be a distance from the proximal end 912 X16 of about 10.2 mm, or in a range of 9 mm to 35 mm. In some embodiments, the position of the guidewire port 911 may be greater than X14+X15.

In some embodiments, the cut pattern 905 may start at a distance from the proximal end of about 10.2 mm or in a range of 10 mm to 12 mm. As previously described, the cut pattern 905 may be made of individual features 906 like the cuts or windows pictured in FIG. 27. Each of the features 906 or spacing between features 906 may vary. In some embodiments, the features may have a width (e.g., cut width) X10 of about 0.4 mm, or in a range of 0.35 mm to 0.50 mm. As described herein, in some embodiments, a width between adjacent features 906 may decrease from the proximal end 912 to the distal end 902. The decrease in the width between adjacent features 906 may be gradual, proportional to distance from the proximal end 912, or exponential to the distance from the proximal end 912. For example, width X9 between adjacent features may be smaller than a width X11 between adjacent features. For example, the width X9 can be about 0.74 mm and the width X11 can be about 1.11 mm. In some embodiments, a distal pitch X18 can be about 3.5 mm, or a range of 3 mm to 5 mm. The pitch of the features 906 may be consistent along the length of the inlet tube.

In some embodiments, the inlet device 1000 may be connected to other components of an MCS system or device by an interference fit. For example, the proximal end 912 can be coupled to a motor housing, or an impeller housing, or an impeller bearing via an interference fit. The distal end 902 can be coupled to a distal tip by an interference fit.

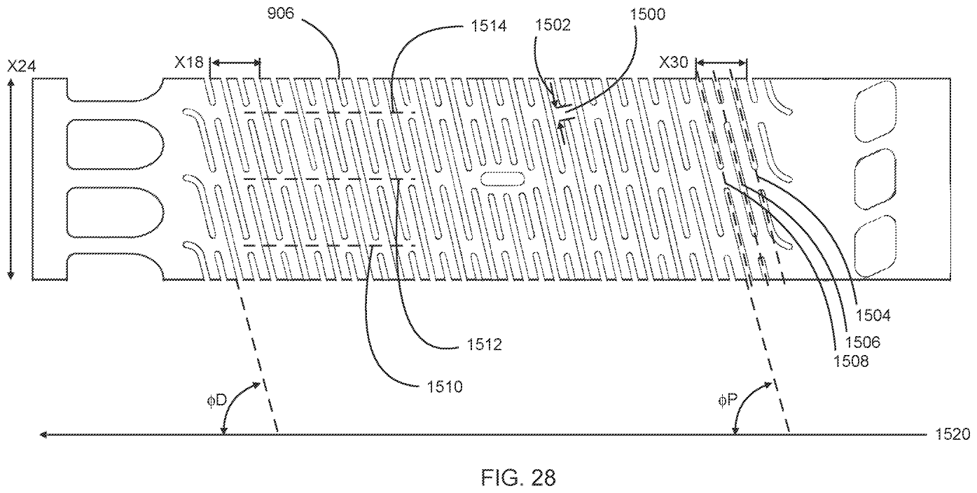

Referring to FIG. 28, a representation of the cut pattern 905 on an inlet device 1000 as shown in FIG. 27 is shown. As illustrated in FIG. 27, a three-dimensional tubular inlet device 1000 can be represented by an unrolled two-dimensional rectangle having a width equal to the circumference X24. In some embodiments, the features 906 of the inlet device 1000 may be linear slots or cuts that are oriented at an acute angle f to the longitudinal axis 1520. The acute angle f may be in a range of about 60 degrees to 80 degrees (e.g., in a range of 65 to 80 degrees, about 73 degrees). The acute angle f may be consistent with all of the features 906.

In a tubular form, the angled features 906 on the inlet device 1000 may create a helical pattern with periodic interruptions 1500 (i.e., areas where a helical feature is interrupted by solid material before another feature continues along the same helical path). The periodic interruptions may occur at a frequency of 3 times per 2 revolutions around the inlet device 1000. The length 1502 of the interruptions may be about 1.5 mm, or in a range of 1 to 3.5 mm, and may be consistent for each interruption. The cut pattern 905 may include a first plurality of features 906 aligned on a first helical path 1504, wherein the first plurality of features 906 are separated from one another by interruptions 1500. The cut pattern 905 may further include a second plurality of features 906 aligned on a second helical path 1506. The cut pattern 905 may further include a third plurality of features 906 aligned on a third helical path 1508.

The plurality of helical paths (e.g., two or three or more) may be adjacent to one another and travel in the same direction around the inlet device 1000 and be separated from one another by a bar width. In other words, the second and third helical paths may be positioned, and optionally equally spaced, within one pitch of the first helical path. Pitch X18 may be a distance along a longitudinal axis between one revolution of a helical path. The interruptions 1500 may be aligned along one of three longitudinal lines 1510, 1512, 1514, that are parallel to the longitudinal axis 1520 and spaced around the circumference of the inlet device. In some embodiments, the spacing may be equal, for example, three longitudinal lines may be 120 degrees from one another around the circumference. In some embodiments, the spacing may be unequal to impart a preferred bending direction. In other embodiments, the interruptions 1500 may be aligned along one longitudinal line, two longitudinal lines, four longitudinal lines or any other suitable number of longitudinal lines.

In some embodiments, the interruptions 1500 may have a longer length 1502 toward the proximal end 912 and longitudinally adjacent interruptions (i.e., interruptions aligned on a longitudinal line 1510, 1512, 1514, may gradually decrease in length in a distal direction for at least a portion of the full length of the inlet device 1000, for example along the length X3 of the proximal section. The gradual decrease in interruption length may be a linear decrease that is proportional to distance from the proximal end 912. This gradual decrease in interruption length can be a factor in creating an inlet device 1000 with a gradually increasing stiffness from the proximal to distal ends.

In some embodiments, the acute angle f may gradually increase from a proximal acute angle fP at the proximal end 912 to a distal acute angle fD at the distal end 902 of the inlet device 1000. For example, proximal acute angle fP may be about 72 degrees and the acute angle f may gradually increase along the longitudinal axis 1520 to a distal acute angle fD of about 76 degrees. The acute angle f may increase by an amount in a range of about 3 to 5 degrees from the proximal acute angle fP to the distal acute angle fD. This gradual increase in acute angle f can be a factor in creating an inlet device 1000 with a gradually increasing stiffness from the proximal end 912 to the distal end 902.

In some embodiments, bar width may decrease distally. For example, X9 may be smaller than X11 and the decrease may be gradual, for example a linear decrease with respect to distance along the longitudinal axis 1520. This gradual decrease in bar width can be a factor in creating an inlet tube with a gradually increasing stiffness from the proximal to distal ends.

In some embodiments, pitch may decrease gradually (e.g., linearly) along the longitudinal axis in a distal direction. For example, a proximal pitch X30 may be larger than a distal pitch X18. The proximal pitch X30 may be about 5.51 mm and the distal pitch X18 may be about 4.91 mm. This gradual decrease in pitch can be a factor in creating an inlet device with a gradually increasing stiffness from the proximal end 912 to the distal end 902.

In some embodiments, coating or covering 915 thickness or number of layers may decrease distally which can be a factor in creating an inlet device with a gradually increasing stiffness from the proximal end 912 to the distal end 902.

In some embodiments, one or a combination of gradually changing interruption length 1502, acute angle f, bar width, pitch, or covering thickness may be utilized to make an inlet tube with a gradually increasing stiffness from the proximal end 912 to the distal end 902 to reduce a risk of kinking.

Any of the features described herein with respect to FIGS. 1-28 may be used with a variety of different MCS systems and devices, and vice versa. Such different MCS systems and devices include, for example, those as described in U.S. provisional application No. 63/116,616, filed Nov. 20, 2020 and titled Mechanical Left Ventricular Support System for Cardiogenic Shock, in U.S. provisional application No. 63/116,686, filed Nov. 20, 2020 and titled Mechanical Circulatory Support System for High Risk Coronary Interventions, in U.S. provisional application No. 63/224,326, filed Jul. 21, 2021 and titled Guidewire, in international PCT applications no. PCT/EP2019/076002 filed Sep. 26, 2019 and titled Sealed Micropump, in PCT/EP2019/062731 filed May 16, 2019 and titled Permanent-magnetic radial rotating joint and micropump comprising such a radial rotating joint, in PCT/EP2019/062746 filed May 16, 2019 and titled Rotor bearing system, in PCT/EP2019/064775 filed Jun. 6, 2019 and titled Line device for a ventricular assist device and method for producing a line device, in PCT/EP2019/064780 filed Jun. 6, 2019 and titled Sensor head device for a minimal invasive ventricular assist device and method for producing such a sensor head device, in PCT/EP2019/064136 filed May 30, 2019 and titled Line device for conducting a blood flow for a heart support system, and production and assembly method, in PCT/EP2019/064807 filed Jun. 6, 2019 and titled Method for determining a flow speed of a fluid flowing through an implanted, vascular assistance system and implantable, vascular assistance system, in PCT/EP2019/071245 filed Aug. 7, 2019 and titled Device and method for monitoring the state of health of a patient, in PCT/EP2019/071233 filed Aug. 7, 2019 and titled Bearing device for a heart support system, and method for rinsing a space in a bearing device for a heart support system, in PCT/EP2019/068434 filed Jul. 9, 2019 and titled Impeller housing for an implantable, vascular support system, in PCT/EP2019/069571 filed Jul. 19, 2019 and titled Feed line for a pump unit of a cardiac assistance system, cardiac assistance system and method for producing a feed line for a pump unit of a cardiac assistance system, and/or in PCT/EP2019/075662 filed Sep. 24, 2019 and titled Method and system for determining a flow speed of a fluid flowing through an implanted, vascular assistance system, the entire disclosure of each of which is incorporated by reference herein for all purposes and forms a part of this specification and description.

If an exemplary embodiment comprises a “and/or” link between a first feature and a second feature, this is to be read in such a way that the embodiment according to one embodiment has both the first feature and the second feature and according to a further embodiment has either only the first feature or only the second feature.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within ±15% of, within ±10% of, within ±5% of, within ±1% of, within ±0.1% of, and within ±0.01% of the stated amount, or other ranges depending on context and as may be understood by one of ordinary skill in the art. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. An inlet device for use in a mechanical circulatory support system, the inlet device comprising:
an inlet portion for admitting a body fluid of a patient into the inlet device; and
a transfer portion connected to and in fluid communication with the inlet portion and having a support structure, wherein the transfer portion is deformably formed.

2. An inlet device as in claim 1, wherein the transfer portion comprises a transition portion adjacent to and in fluid communication with the inlet portion.

3. An inlet device as in claim 2, wherein the transition portion comprises a variable cross-sectional area and/or variable diameter.

4. An inlet device as in claim 3, wherein the variable cross-sectional area and/or variable diameter of the transition portion increases away from the inlet portion.

5. An inlet device according to claim 1, wherein a cross-sectional area and/or diameter of the transfer portion is greater than a cross-sectional area and/or diameter of the inlet portion.

6. An inlet device according to claim 5, wherein the cross-sectional area and/or the diameter of the transfer portion is 25% to 35% greater than the cross-sectional area and/or diameter of the inlet portion.

7. An inlet device according to claim 1, wherein an inner and/or an outer surface of the transfer portion comprises a polymer coating and/or a polymeric sleeve.

8. An inlet device according to claim 1, wherein the support structure of the transfer portion is formed from a plurality of serrated and/or zig-zagged structural elements.

9. An inlet device according to claim 8, wherein the serrated and/or zig-zagged structural elements are arranged substantially obliquely to a longitudinal axis of the transfer portion and comprise finger elements.

10. An inlet device according to claim 1, wherein the support structure of the transfer portion is formed from a plurality of structural elements arranged substantially obliquely to a longitudinal axis of the transfer portion.

11. An inlet device according to claim 1, wherein the support structure of the transfer portion is at least partially wound.

12. An inlet device according to claim 1, wherein the inlet device is at least partially wound.

13. An inlet device according to claim 1, wherein the support structure comprises a super-elastic material.

14. An inlet device according to claim 13, wherein the super-elastic material comprises Nitinol.

15. An inlet device according to claim 1, wherein the inlet device comprises a super-elastic material.

16. An inlet device according to claim 15, wherein the super-elastic material comprises Nitinol.

17. An inlet device according to claim 1, wherein the support structure prevents collapse of the transfer section while a body fluid of a patient is transferred through the inlet device.

18. An inlet device according to claim 1, wherein the body fluid is blood.

19. An inlet device according to claim 1, wherein the transfer portion comprises a plurality of cuts configured to increase flexibility of the transfer portion.

20. An inlet device according to claim 19, wherein a width between adjacent cuts decreases from a proximal section of the inlet device to a distal section of the inlet device.

21. A connecting device for attaching and/or detaching a component of a mechanical circulatory support system, comprising:
a receiving element with a receiving structure; and
an insertion element that can be pushed into the receiving structure of the receiving element, the insertion element comprising at least one slide-on ramp, the slide-on ramp being connectable to the receiving structure of the receiving element in a form-fitting, non-positive, force-locking, and/or self-locking manner.

22. A connecting device as in claim 21, wherein the receiving element and/or the insertion element is formed substantially cylindrically as a tube.

23. A connecting device according to claim 21, wherein the receiving structure comprises at least one through-opening.

24. A connecting device as in claim 23, wherein in the assembled state, the at least one slide-on ramp of the insertion element locates within the at least one through-opening of the receiving structure of the receiving element.

25. A connecting device according to claim 21, wherein in the assembled state, an expansion joint is formed between the receiving element and the insertion element.

26. A connecting device according to claim 21, wherein the receiving structure comprises a wave-like longitudinal edge.

27. A connecting device as in claim 26, wherein the insertion structure comprises a raised wave-like feature on its outer surface complementary to the wave-like longitudinal edge of the receiving structure.

28. A connecting device according to claim 21, wherein the receiving element and/or the insertion element is comprised at least in part of a super-elastic material.

29. A connecting device as in claim 28, wherein the super-elastic material comprises Nitinol.

30. A connecting device according to claim 21, wherein the receiving element and/or the insertion element is comprised at least in part of titanium.

31. A method of assembling a connecting device according to claim 21, the method comprising:
providing the receiving element with the receiving structure and the insertion element with the at least one slide-on ramp; and
pushing the insertion element into the receiving structure of the receiving element, wherein the slide-on ramp connects to the receiving structure in a form-fitting, non-positive, force-locking, and/or self-locking manner.

32. A method of assembling a connecting device according to claim 31, wherein the receiving element and/or the insertion element is comprised at least in part of Nitinol, further comprising cooling the connecting device and/or insertion element to a temperature below an austenite transformation temperature of the Nitinol prior to the pushing step.

33. A method of disassembling a connecting device according to claim 21, the method comprising:
providing the receiving element with the receiving structure and the insertion element with the at least one slide-on ramp in a connected configuration, wherein in the connected configuration the at least one slide-on ramp connects to the receiving structure in a form-fitting, non-positive, force-locking, and/or self-locking manner;
applying an inward radial force to the at least one slide-on ramp of the insertion element; and
pulling apart the receiving element and the insertion element.

34. A method of disassembling a connecting device according to claim 33, wherein the receiving element and/or the insertion element is comprised at least in part of Nitinol, further comprising cooling the connecting device to a temperature below an austenite transformation temperature of the Nitinol prior to the pulling apart step.
